(19) United States
(12) Patent Application Publication
(10) Pub. No.: US 2002/0166760 A1
(43) Pub. Date: Nov. 14, 2002

(54) Micromagentic systems and methods for microfluidics

(76) Inventors: Mara G. Prentiss, Cambridge, MA (US); Gary Zabow, Somerville, MA (US); Tao Deng, Cambridge, MA (US); Mala Lakshmi Radhakrishnan, San Jose, CA (US); George M. Whitesides, Newton, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/997,998

(22) Filed: Nov. 30, 2001

Related U.S. Application Data
(63) Continuation-in-part of application No. 09/853,888, filed on May 11, 2001.

(51) Int. Cl. C25B 5/00; G01N 33/553

(52) U.S. Cl. 204/155; 436/526

(57) ABSTRACT

The invention provides systems and methods of manipulating biological or chemical species. The species may be attached to a magnetic particle which is manipulated using micro-magnetic fields. In some cases, the magnetic fields are generated by current carrying wires that are patterned on a substrate. The magnetic fields define channels on the surface of the substrate in which the magnetic particles and attached species may be transported, positioned and stored. In other cases, the magnetic fields are generated by magnetic features located within the channels on the surface of the substrate. Thus, the systems and methods can manipulate biological or chemical species on a microscale (e.g., less than 5 cm). Applications of the systems and methods are in, but are not limited to, the fields of biotechnology, microanalysis, and microsynthesis.

### RELATED APPLICATION

This application is a continuation-in-part of co-pending U.S. application Ser. No. 09/853,888, “Micromagnetic Systems and Methods for Microfluidics”, filed May 11, 2001, which is incorporated herein by reference.

### FIELD OF THE INVENTION

The invention relates generally to micromagnetic systems and methods and, more particularly, to systems and methods which manipulate biological or chemical species using magnetic fields in microfluidic applications.

### BACKGROUND OF THE INVENTION

The ability to manipulate chemical species (e.g., chemical reagents) or biological species (e.g., cellular material, polymers, proteins, DNA, and the like) on a microscale is important in many applications. Such applications are in the fields of biotechnology, microanalysis, and microsynthesis, amongst others. Depending on the application, the manipulations may involve separating, transporting, positioning, and/or storing the species.

Conventionally, microfluidic systems can be used to manipulate chemical or biological species. Microfluidic systems have been previously described, for example, in: McDonald J. C., D. C. Duffy, J. R. Anderson, D. T. Chiu, H. K. Wu, O. J. A. Schuller, and G. M. Whitesides, Electrophoresis, 21(1): 27-40, Jan, 2000. These systems involve controlling fluid flow on a microscale. Chemical or biological species that are suspended in the fluid may, thus, be manipulated. In some microfluidic systems, pumps and/or valves are used to control fluid flow through a series of physical microchannels formed within a substrate. Such systems generally are not easily fabricated, have a complex structure, and are not easily reconfigured for different operations or dynamically.

Accordingly, a need exists for systems and methods for manipulating chemical or biological species which overcome one or more of the disadvantages of the conventional techniques.

### SUMMARY OF THE INVENTION

The invention provides systems and methods of manipulating biological or chemical species. The species may be attached to a magnetic particle which is manipulated using micro-magnetic fields. In some cases, the magnetic fields are generated by current carrying wires that are patterned on a substrate. In other cases, the magnetic fields are generated by magnetic features located within the channels on the surface of the substrate. The magnetic fields define channels on the surface of the substrate in which the magnetic particles and attached species may be transported, positioned, and stored amongst other operations. Thus, the systems and methods can manipulate biological or chemical species on a microscale. Applications of the systems and methods are in, but are not limited to, the fields of biotechnology, microanalysis, and microsynthesis.

In one aspect, the invention provides a method of manipulating a biological or a chemical species. The method includes manipulating a biological or a chemical species in a confined space having a maximum dimension of less than 5 cm using a magnetic field.

In another aspect, the invention provides a method of manipulating a biological or chemical species. The method includes manipulating a biological or a chemical species on a substrate in the absence of structural boundaries capable of confining the species.

In another aspect, the invention provides a method of manipulating a biological or a chemical species. The method includes manipulating a biological or a chemical species using a magnetic field generated by one or more current carrying wires.

In another aspect, the invention provides a method of manipulating a biological or a chemical species. The method includes moving a biological or chemical species in a first direction, and changing the direction of motion of the biological or chemical species using a magnetic field.

In another aspect, the invention provides a method of manipulating a biological or a chemical species. The method includes manipulating a biological or a chemical species on a substrate in the absence of fluid flow.

In another aspect, the invention provides a microfluidics system. The system includes a substrate including a plurality of wires capable of carrying current to generate magnetic fields that define channels on the substrate, and a biological or chemical species movable within the channels on the substrate.

In another aspect, the invention provides a microfluidics system. The system includes a channel and a feature formed within the channel. The feature is capable of generating a magnetic field.

In another aspect, the invention provides a microfluidics system. The system includes a substrate having a channel defined therein, and a feature formed within the channel. The feature comprises a magnetic material.

In another aspect, the invention provides a method of manipulating a chemical or biological species within a channel in a microfluidics system, using a magnetic field generated by a feature formed within the channel.

In another aspect, the invention provides a method of generating a magnetic field confined within a microfluidic channel.

In another aspect, the invention provides a method of forming a microfluidics system. The method includes the steps of forming a channel defined by a microfluidics system, and forming a feature within the channel. The feature is capable of generating a magnetic field.

In another aspect, the invention provides a microfluidics system. The system includes a channel, and a feature having a smallest dimension no greater than the smallest dimension of the channel proximate the feature. The feature is positioned so as to be capable of generating a magnetic field within the channel.

In another aspect, the invention provides a method of applying a magnetic field able to manipulate a chemical or biological species within a first channel in a microfluidics system, where the magnetic field is unable to manipulate a similar species within a second channel in the microfluidics system.

Amongst other advantages, the systems and methods of the invention permits manipulation of chemical or biological species on a microscale (e.g., less than 5 cm). Microscale applications are particularly well-suited because the resulting high magnetic field gradients generates large net forces on the chemical or biological species (or magnetic particles attached thereto). Furthermore, in some embodiments, such systems can manipulate species without the need for complex pumps and/or valves to control fluid flow. Also, substrates of the systems may be easily fabricated using conventional lithography techniques. The systems may also be easily re-configured, for example, by changing the current flow in wires patterned on the substrate to define different channels in which the species are manipulated. It is even possible to reconfigure the system during use (e.g., in real-time), for example, in response to measurements made by the system.

Other advantages, aspects, and features of the invention will become apparent from the following detailed description of the invention when considered in conjunction with the accompanying drawings. It should be understood that not every embodiment of the invention will include all of the advantages described herein.

### BRIEF DESCRIPTION OF THE DRAWINGS

FIG. 1 is a schematic plan view of a micromagnetic system that includes a microchip substrate according to one embodiment of the present invention.

FIG. 2 illustrates the resulting magnetic field at a selected height above parallel wires which carry current in opposite directions.

FIG. 3 illustrates the transport of magnetic particles through a channel defined by magnetic fields according to one embodiment of the present invention.

FIGS. 4A to 4D illustrate the movement of a magnetic particle by varying magnetic fields according to one embodiment of the present invention.

FIG. 5 illustrates a biological or chemical species attached to a magnetic particle according to one embodiment of the present invention.

FIGS. 6A-6D illustrate the steps of forming a microchip using the soft lithography process of the Example.

FIG. 7 schematically illustrates the micromagnetic system used in the Example.

FIGS. 8A to 8C show the confinement of magnetic beads in the Example using a magnetic field.

FIG. 9 illustrates a micromagnetic system in one embodiment of the invention.

FIG. 10 illustrates another embodiment of the invention, showing an array of features.

FIG. 11 illustrates a side view of an embodiment of the invention, showing an array of features.

FIG. 12 illustrates a fabrication method of an embodiment of the invention.

FIGS. 13A to 13C are a series of SEM photomicrographs of one embodiment of the invention.

FIGS. 14A to 14C are a series of optical photomicrographs of one embodiment of the invention.

FIG. 15 illustrates an embodiment of the invention having a tubular substrate.

### DETAILED DESCRIPTION

The invention provides systems and methods for manipulating biological or chemical species. The species are manipulated on a microscopic scale using magnetic fields.

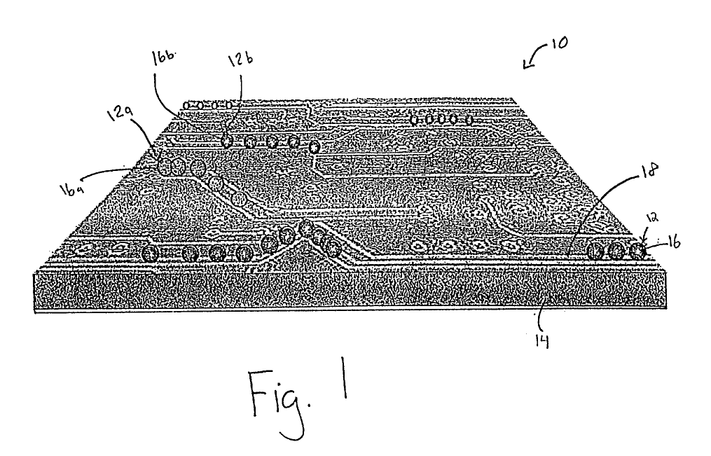

FIG. 1 shows a micromagnetic system 10 for manipulating chemical and biological species 12 on a substrate 14 according to one embodiment of the invention. Species 12 is attached to a magnetic particle 16. As shown, system 10 includes multiple particles 16 which, for example, are dispersed in a fluid medium disposed on the surface of substrate 14. System 10 generates localized magnetic fields by passing current through wires 18 formed on substrate 14. The magnetic fields are used to manipulate particles 16 and, consequently, species 12 attached thereto. Substrate 14 typically includes a pattern of multiple wires 18. Current flow through wires 18 is controlled to manipulate the species on system 10 as desired. The pattern of wires 18 enables multiple types of manipulation and allows for simple reconfiguration of the system as described further below.

Species 12 are manipulated using the principle that particles 16 are attracted to magnetic fields and, particularly, to locations where relatively strong magnetic fields compared to immediate surrounding regions (i.e., local magnetic field maxima) are present. Current passing through a wire, or an arrangement of wires, can generate a magnetic field. The magnitude and location of the magnetic field generated depends, in part, upon system design parameters (e.g., the wire arrangement) and system operating parameters (e.g., the amount of current). Another way to generate a magnetic field is using an externally applied magnetic field. In some embodiments, both magnetic fields generated by current carrying wires and externally magnetic fields may be used. System 10 is designed and operated in a manner that generates localized magnetic fields in specific locations which attract and manipulate particles 16, as described further below.

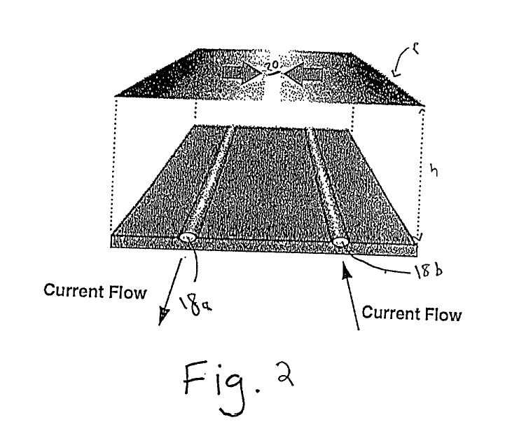

FIG. 2 schematically illustrates one portion of substrate 14 which includes parallel wires 18a, 18b that may be utilized on substrate 14 to generate a magnetic field according to one embodiment of the invention. When parallel wires 18a, 18b carry current in the opposite direction (as indicated by arrows), a magnetic field is generated above substrate 14. The magnetic field has a magnitude in plane (p) that is proportional to the degree of shading (i.e., regions of strong magnetic field are lightly-shaded and regions of weak magnetic field are darkly-shaded). In this illustrative arrangement, the strongest magnetic field in plane (p) is located in a region 20 equidistant between current carrying wires 18a, 18b. It should also be understood that system 10 may include other arrangements of wires to generate magnetic fields including single wires and wires with one or more turns.

Referring to FIG. 3, substrate 14 includes an arrangement of parallel current carrying wires 18a, 18b similar to the arrangement shown in FIG. 2. The magnetic field generated by current carrying wires 18a, 18b attracts magnetic particles 16 and confines the particles to a region between the wires 18a, 18b where a local magnetic field maxima is present. Such regions of strong magnetic field, therefore, define a channel 22 that extends between parallel wires 18a, 18b.

Channels 22 may have a variety of different dimensions as required for a particular application of system 10. Typical channel widths are less than about 500 microns. In other cases, shorter channel widths are desired such as widths of less than about 100 microns or less than about 50 microns. Shorter channel widths may be desired, for example, when the pattern includes a large number of channels. Generally, channel lengths are less than about 5 cm. More typically, even shorter channel lengths are utilized such as less than about 5 mm, or even less than about 0.5 mm. In some embodiments, system 10 may include a number of channels 22 which have different lengths and/or widths. Channels 22 may have different shapes which include tapered channels and or channels with enlarged regions. In some cases, channel 22 include a closed end that defines, for example, an enlarged region that may have a width greater than that of the channel. Such enlarged regions may be for storage of the species.

Wires 18 may be formed on substrate 14 in any variety of patterns and the particular pattern may be designed for the desired application. In one set of embodiments, wires are formed in a grid pattern. Current flow through the pattern of wires 18 is controlled so as to selectively form channels 22 in specific locations when desired. In many cases, multiple channels 22 may be formed at the same time by simultaneously passing current through different wires (or wire arrangements) on substrate 14. However, it should be understood that current may not flow through all wires 18 patterned on substrate 14 at all times. Because channel formation is controlled by current flow, substrate 14 may be easily re-configured to provide different channels by changing which wires 18 in the pattern carry current.

The confinement of particles 16, and species 12 attached thereto, within channels 22 is one type of manipulation provided by the present invention. In some cases, particles 16 are further manipulated once confined within channels 22. For example, particles 16 may be transported within channels 22. If particles 16 are suspended in a fluid medium, the particles may be transported within channel 22 via attraction by another magnetic field (i.e., a field that is different than the field that formed the channel). The magnetic field which attracts the particles within the channel may be generated by a current carrying wire positioned nearby or an external magnetic field. When a current carrying wire is used to generate the field that attracts the particles, the current may be pulsed so as to limit heating. In other cases, as described further below, particles 16 may be transported by varying the location of the local field maxima within channel 22.

Wires 18 may be patterned in a manner that forms channels 22 which can transport particles 16 to a desired location (e.g., a storage region). Once transported to the desired location, species 12 attached to particles 16 may be stored, detected, caused to react with another species, or otherwise further manipulated. Channels 22 advantageously permit transportation of species 12 without structural boundaries, such as physical channels which are formed in the substrate, as in certain conventional microfluidic systems. In some cases, channels 22 are used in conjunction with physical channels to enhance performance.

In certain embodiments, particles 16 may be transported within channel 22 by varying the position of the local field maxima. As described above, particles 16 are attracted to a position where strong magnetic fields are present. Thus, by appropriate changing the position of the strongest magnetic field, particle 16 may be moved.

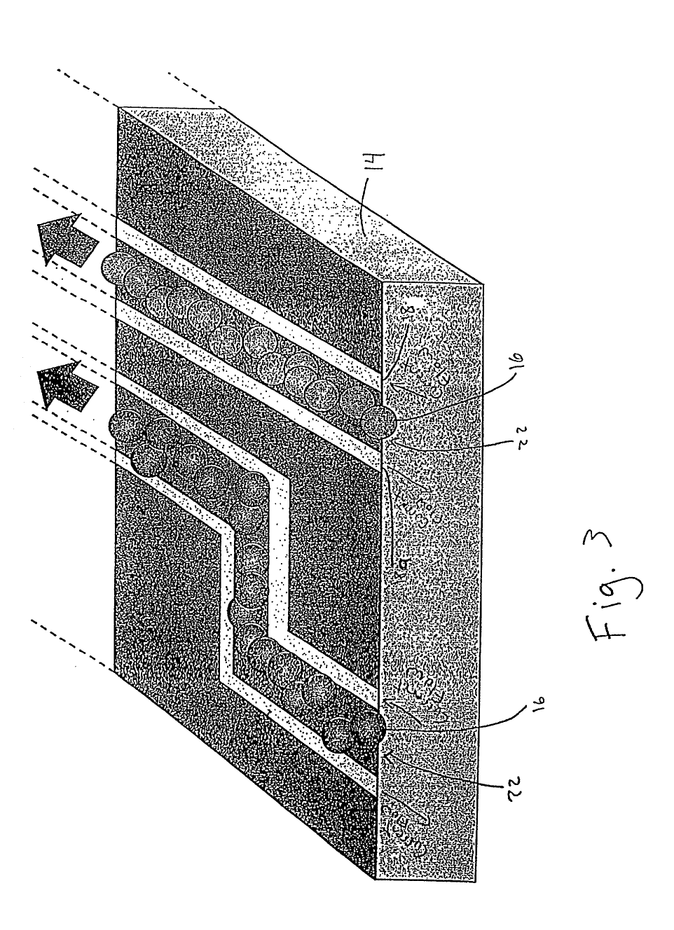

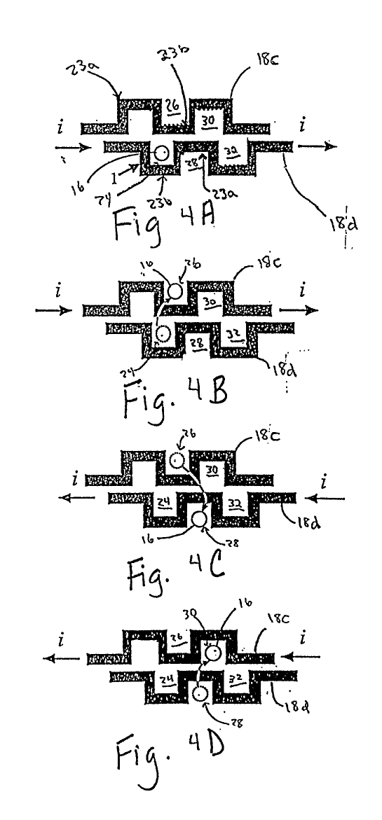

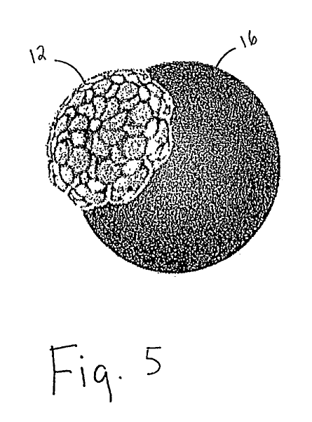

FIGS. 4A to 4D schematically illustrate transporting particle 16 by moving the position of the local field maxima generated by current flowing through wires 18c, 18d in combination with an applied bias field. Wires 18c, 18d have a series of turns including an alternating arrangement of n-shaped turns 23a and unshaped turns 23b. Depending on the direction of current flow and the direction of the applied bias field, the location of the local field maxima is in a position (e.g., 24, 26, 32) within unshaped turns 23b, or a position (e.g., 28, 30) within n-shaped turns 23a. By selectively varying which of wire 18c, 18d carries current and the direction of the current, the position of the local field maxima and particle 16 may be moved.

In FIG. 4A, particle 16 is confined to a position 24 within u-shaped turn 23b, where the local field maxima is generated from current flowing downstream (shown by arrow) through wire 18d. In FIGS. 4A to 4D, a bias field is applied in a perpendicular direction coming out of the page. To transport particle 16, current flow through wire 18d is stopped and downstream current flow through wire 18c is started. The local field maxima is now generated at a position 26 within u-shaped turn 23b causing particle 16 to move from position 24 to position 26 (FIG. 4B). To continue the transportation of particle 16, current flow through wire 18c is stopped and upstream (shown by arrow) current flow through wire 18d is started. The local field maxima is now generated at a position 28 causing particle 16 to move from position 26 to position 28. To continue the transportation of particle 16, the current flow through wire 18c is stopped and upstream current flow through wire 18d is started. The local field maxima is now generated at a position 30 causing particle 16 to move from position 28 to position 30 (FIG. 4D). In this manner, particle 16 (or a plurality of particles) may be transported by moving the location of the local field maxima.

In the methods of the invention, the magnetic field generated by current carrying wires 18 should have a maximum value sufficient to attract particles 16. In some embodiments, the strongest field generated by current carrying wires 18 is less than about 2 kG (e.g., on the order of about 1 kG). Different applications may require different field strengths. The magnetic fields generated by current carrying wires 18 generally act over a short range. For example, the fields may be localized to act over a range of less than about 100 microns. The localization permits confinement of particles 16 within small dimensions which enables a number of processes to occur in parallel on the same substrate. The magnetic fields may also be easily adjusted, controlled, or reconfigured, by changing the amount of current flow, the direction of current flow, or which wires carry current. System 10, thus, is very flexible and can be easily tailored for different applications.

In one embodiment, wires 18 have a width between about 50 microns and about 100 microns and a height between about 10 microns and about 20 microns. Wires 18 having such dimensions are generally capable of carrying direct current of at least about 10 A at room temperature which can generate maximum magnetic fields on the order of about 1 kG. It should be understood that wires 18 may also have other dimensions if desired for a particular application.

Wires 18 may be fabricated using known lithography techniques on the surface of the substrate 14 including soft lithography techniques. Suitable lithography techniques typically include deposition, patterning, and etching steps to form wires having the desired arrangement. An exemplary lithography technique has been described in Xia YN. Whitesides GM. SOFT LITHOGRAPHY. [Review]. Angewandte Chemie (International Edition in English). 37(5):551-575, 1998 Mar 16., which is incorporated herein by reference.

In some embodiments, an external magnetic field (i.e., a bias field) may be superimposed on the field generated by the current carrying wires. Superimposed external fields may be used, for example, to change patterns of local magnetic field maxima by constructively and/or destructively interfering with the field generated by the current carrying wires. The external magnetic field can be generated by an external magnet positioned proximate to substrate 14.

In some embodiments, system 10 optionally includes a magnetic material layer 31 (FIG. 1) formed on substrate 14. Magnetic material layer 31, for example, may be formed between wires 16 and substrate 14 (i.e., wires 16 are formed on magnetic material layer) or on top of wires 16. It should be understood that, in other embodiments, system 10 may not include a magnetic material layer 31. Magnetic material layer 31 comprises a magnetic material in which a magnetic field may be induced semi-permanently. That is, a field induced in the material is retained in the material (even when the inducing field is removed), and the induced field can be erased by an another applied field. Examples of such magnetic materials include compounds (e.g., oxides) of cobalt, iron, and chrome.

When magnetic material layer 31 is used, fields generated by current carrying wires 16 include local magnetic field maxima within magnetic material layer 31. The local magnetic field maxima generated by the magnetic material layer defines, in part, channels 22 in conjunction with the local magnetic field maxima generated by the current carrying wires. Even when current flow through wires 16 is stopped, the local field maxima continue to be generated by magnetic material layer 31 and continue to define channels 22. Thus, channels 22 can be formed by applying the current for a short time (i.e., pulsing the current) to induce local field maxima in magnetic material layer 31. The pulsing of the current may advantageously reduce heating effects associated with current flowing through wires for long time periods. Thus, utilization of magnetic material layer 31 may be preferred in some systems that are particularly susceptible to damage from over heating. The induced local field maxima in magnetic material layer 31 may be removed by applying an external field of sufficient strength and opposite direction, for example, in order to re-configure channels 22 within system 10.

Species 12 can be any biological or chemical species. Typical examples include chemical reagents, cellular material, nucleic acids, proteins, polypeptides, lipids, carbohydrates, and polymers including synthetic polymers. In some applications, more than one type of species 12 may be manipulated at the same time using micromagnetic system 10. Different types of species 12 may be attached to different particles. As shown in FIG. 1, system 10 may be used to manipulate, in parallel operations, a first species 12a attached to a particle 16a and a second species 12b attached to a particle 16b. However, it should be understood that it is also possible for an individual particle 16 to have more than one type of species attached thereto.

Magnetic particle 16 may have any composition that enables it to be manipulated by a magnetic field. The composition typically includes at least one magnetic component and also may include one or more non-magnetic components. In some embodiments, magnetic particle 16 may comprise a superparamagnetic material (i.e., materials that lose their magnetization in the absence of a magnetic field) which may allow for recycling of particles. In some cases, particle 16 has a non-magnetic coating around a magnetic core. The coating may have a chemical structure that permits attachment of species 12 thereto. Species 12, for example, can be chemically bonded to the coating thereby attaching the species to the particle. Suitable coatings include polymeric materials, such as polystyrene. The particular coating composition can depend upon the type of species 12 being attached.

Particles 16 may have a variety of shapes and sizes depending on the application. In some embodiments, a substantially spherical particle (i.e., a bead) may be preferred. In most microscale applications, the size of particles 16 are less than 100 microns. However, larger size particles may also be used if desired. In some embodiments, the particle size is less than about 10 microns; in others, the particle size is less than about 1 micron; in others less than about 100 nanometers. In some embodiments the particle size is between about 1 micron and about 10 microns. Smaller particle sizes may be desired, for example, in systems that have small channel widths.

System 10 may utilize one type of particle 16 (i.e., same composition and dimensions), or may utilize more than one type of particle. Different types of particles may be used in system 10, for example, if more than one type of species 12 is being manipulated. However, it should also be understood that one type of particle may be used with different species.

In some embodiments, it is desirable for species 12 to be selectively attached to particle 16. That is, species 12 can be attached to particle 16 under certain conditions and can be released from particle 16 under other conditions. For example, species 12 may be attached to particle 16 at the start of an operation and then transported to another position on substrate and released from the particle. Species 12 may be attached to particle 16 through chemical bonding via a reaction between the species and a component of the particle (e.g., a coating on particle 16) and released by removing the bond, for example, using a solvent. The solvent may be introduced into system 10 at a desired location to release the species. Once released, species 12 may react with other species or be analyzed, amongst other operations.

In some preferred cases, particles 16 are dispersed in a fluid (not shown) disposed on the surface of substrate 14. Suitable fluids include water and non-aqueous fluids, as well as mixtures and solutions thereof. Additives may be added to the fluid to promote dispersion or for other reasons. The fluid can provide a low friction medium in which particles 16 may be manipulated. In these embodiments, particles 16 may be manipulated irrespective of fluid flow. In some cases, no fluid flow occurs on substrate 14. In certain cases, however, fluid flow may be used to enhance particle manipulation. It should also be understood that particles 16 may not be dispersed in a fluid in certain embodiments.

Substrate 14 may be any suitable substrate. For example, substrate 14 may be any type used in integrated circuit applications such as a microchip. Suitable substrate materials include semiconductor (e.g., silicon) materials and polymeric materials. Substrate 14 may have a number of layers formed thereupon including oxide layers, metallic layers (which may be magnetic layers), and the like. The dimensions of substrate 14 may be determined, in part, by the application. In some cases, the surface area of substrate is less than about 10 cm²; in others less than about 1 cm²; and in others less than about 1 mm². The maximum dimension (e.g., length or width) of substrate 14 may be less than about 5 cm, in other cases less than 5 mm; and, in other cases, less than 1 mm.

Particles 16 are manipulated in the systems and methods of the invention in any number of different ways. For example, the magnetic fields may be used to manipulate particles 16 by directing, transporting, storing, positioning, trapping, confining, separating, and mixing, amongst other types of manipulation. In exemplary cases, biological or chemical species 12 are transferred between storage microcells, reaction microcells, or detection microcells. The particular manner in which magnetic particles 16 are manipulated depends upon the application of system 10. Manipulation system 10 may be used in any number of applications. Because system 10 uses magnetic fields on a microscopic level, large numbers of manipulations may be provided on a single substrate 14. Thus, a large number of different operations may occur in parallel on system 10. Also, because system 10 involves manipulating species on the microscopic scale, operations can occur within short time periods.

It should be understood that the systems and methods of the invention may have a variety of variations. For example, the micromagnetic fields may be generated using techniques other than current carrying wires. Other variations will be known to one of ordinary skill in the art.

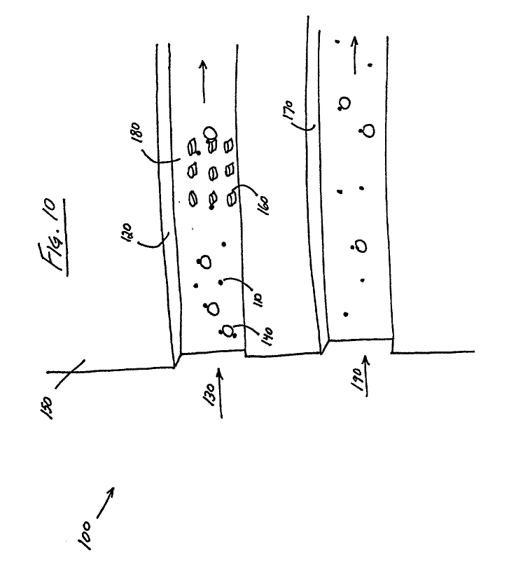

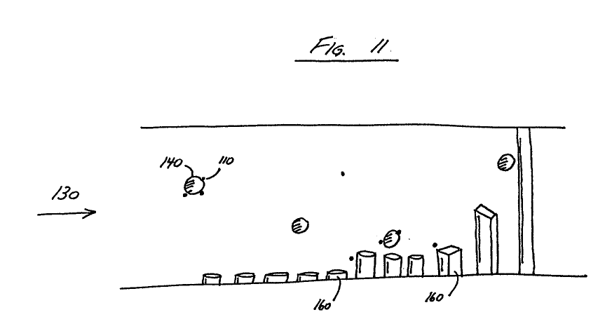

FIGS. 9 and 10 illustrate microfluidic systems 100 for manipulating a chemical or biological species 110, using a magnetic field according to another embodiment of the invention. In the embodiment of FIG. 10, species 110 are attached to particles 140, that are dispersed in a fluid 130 within channels 120 formed in a substrate 150. As described further below, a magnetic feature 160 formed within the channels generates localized magnetic fields that manipulate the particles and the species attached thereto. Magnetic feature 160 may be, for example, posts that extend from a bottom wall 180 of the channels. The features may comprise a ferromagnetic material such as nickel or neodymium. System 100 may be used to manipulate the species in a number of different ways, including the separation of different types of species 110.

Magnetic feature 160 may be any feature that produces a magnetic field capable of manipulating particles 140 and species 110 attached thereto within channel 120. The magnetic features may have a variety of different structures. In some embodiments, the magnetic features are posts. As used herein, the term “post” refers to any structure that protrudes into a channel. There may be a single post or an array of multiple posts. Arrays of posts may be arranged in a regular pattern, for example, a rectangular or a hexagonal pattern, or the posts may be randomly arranged within channel 120. The posts may all have identical shapes or sizes, or they may have different sizes, shapes, magnetic susceptibilities, compositions, or other physical characteristics. The posts may have any shape, for example, pyramidal, conical, spherical, or amorphous. In some embodiments, the posts are cylindrical. The posts may have cross-sections that are square, U-shaped, circular, triangular, or the like. The posts may span channel 120, or they may be smaller than the size of the channel. The posts may have any suitable dimension. In some cases, the posts have a height of less than about 10 μm and a cross-section of less than about 100 μm. For example, in one embodiment, the post has a height of about 7 μm and a circular cross-section of about 15 μm. In a square fluid channel with a cross-section of 50 μm, most of the flow through the channel is substantially unhindered by posts of these dimensions. The posts may be chosen, for example, to produce an induced magnetic field that is sufficient to trap a specific magnetic particle in a given applied magnetic field. The required magnetic field strength needed will be function of the application. In some cases, the shape of the post may be used to enhance the induced magnetic field strength. For example, a post with a triangular, rectangular, or star-shaped profile may have an enhanced magnetic field near the corners or vertices of the post.

Magnetic features 160 may be located in a number of different positions. For example, features 160 may extend from the floor of channel 120 as illustrated. In other cases, the features may extend from a wall of the channels. In other embodiments, the magnetic features are embedded within walls and/or floors of the channels.

The localized magnetic field may be generated in channel 120 in a number of different ways. In some cases, the localized magnetic field is generated by inducing a magnetic field in magnetic feature 160. For example, a magnetic field may be induced in magnetic feature 160 when the feature comprises a ferromagnetic material by application of a primary magnetic field. Suitable ferromagnetic materials include nickel, neodymium, iron, cobalt, or alloys and mixtures of ferromagnetic materials, such as iron oxides or alnicos.

It should be understood that other techniques also may be used to generate the localized magnetic field within the channel such as producing the field electromagnetically using current carrying wires or using a miniature permanent magnet in or near a wall of channel 120.

In embodiments in which a primary magnetic field is used to induce a field in magnetic feature 160, the primary magnetic field may be produced by any suitable means. For example, the primary field may be induced by a permanent magnet external from substrate 150. In other cases, the primary field is induced by a magnet may be embedded within the substrate, such as from a position near or in channel 120. In some cases, the primary magnetic field may be produced electromagnetically, by applying a current to a wire located near magnetic feature 160. The external magnetic field itself may also be an induced magnetic field.

The magnetic field induced in magnetic feature 160 may be greater than the applied external magnetic field. In some embodiments, the applied external magnetic field is insufficient to manipulate particle 140 and species 110 attached thereto within channel 120, while the magnetic field induced in magnetic feature 160 by the external magnetic field is sufficient to manipulate the particle or species. Lower applied magnetic fields, thus, may be used in some embodiments of the invention. For example, an external magnetic field of less than about 1 kilogauss may be applied; in other cases, the external field is less than about 500 gauss; and, in other cases, less than about 100 gauss.

In some embodiments, magnetic feature 160 may produce a magnetic field localized to channel 120. For example, the magnetic field may be confined within channel 120 without being capable of manipulating particles in other nearby channels, such as in channel 170 in FIG. 10. In certain cases, the magnetic field may also rapidly decrease in field strength with distance away from the magnetic feature. The magnetic field may also be unevenly distributed or otherwise localized within microfluidic channel 120. Localized field gradients may be generated by, for example, unevenly distributing magnetic features 160 within microfluidic channel 120, or by having magnetic features with different shapes or sizes within channel 120. Localized or unevenly distributed magnetic fields may be preferable in some embodiments, for example, when the separation or sorting of multiple particles or species may be desired.

As described above in connection with the embodiments of FIGS. 1-8, species 110 may be any biological or chemical species. In some applications, more than one type of species 110 may be manipulated at the same time using microfluidic system 100. The different types of species 110 may be manipulated in the same channel, or in different channels. Different types of species 110 may be attached to the same particle, or to different particles. Different types of species may be respectively attached to different types of particles. As described further below, system 100 may be used to separate different types of species.

Particle 140 may have any composition that enables it to be manipulated by a magnetic field. Suitable compositions have been described above in connection with the embodiments of FIGS. 1-8. For example, the composition may include at least one magnetic component and may also include one or more non-magnetic components. Particle 140 may have any of the shapes and sizes described above as desired for the particular the application. It may also be desirable for species 110 to be selectively attached to particle 140 as described above. That is, species 110 may be attached to particle 140 under certain conditions and may be released from particle 140 under other conditions. Particles 140 may also be dispersed in a fluid 130.

Fluid 130 may be pumped through microfluidic channels 120 by any suitable means. For example, in some embodiments, capillary action is used to draw fluid through microfluidic channels 120. In other embodiments, fluid 130 is drawn through microfluidic channels 120 using gravitational flow or siphoning techniques. Alternatively, pressure-induced methods to cause fluid flow are used in some embodiments of the invention. For example, a syringe pump or a peristaltic pump may be used to drive fluid through microfluidic channels 120. In still other embodiments, fluid flow is electrically induced. For example, fluids containing charged particles move preferentially in a direction under the influence of an applied electric field. Alternatively, fluid flow may be generated using electroosmotic techniques.

Microfluidic channels 120 may be used to transport species through microfluidic system 100. The channels may include any region within the system through which fluid flows or may be contained within, such as a reaction chamber or “cell.” As used herein, “channel” refers to any region within system 100 through which fluid 130 can flow, including, but not limited to, channels and other passageways, reaction chambers or cells, or the like, for example, as illustrated in FIG. 9. Channels may be sealed at one end, be open at both ends, or may have a plurality of inlets and outlets. Multiple inlets and outlets within channel 120 may be able to handle one fluid or several fluids simultaneously. Channel 120 may further be connected to other components within microfluidic system 100 having other functions.

The microfluidic channels 120 may have a variety of different dimensions, as required for a particular application of microfluidic system 100. Typical channel widths are less than about 500 μm. In other cases, shorter channel widths are desired, such as widths of less than about 100 μm or less than about 50 μm. Shorter channel widths may be desired, for example, when a pattern requires a large number of channels. Generally, channel lengths are less than about 5 cm. Shorter channel lengths may be utilized in some cases, such as channels with lengths of less than about 5 mm, or less than about 0.5 mm. In some embodiments, system 100 may include a number of channels 120 which have different lengths or widths.

Microfluidic channels 120 may also have different shapes or configurations, which include tapered channels or channels with enlarged regions. In some cases, channel 120 may include a closed end that defines, for example, an enlarged region that may have a width greater than that of the length of the channel. Such enlarged regions may be used, for example, storing or temporary holding of species 110.

It should be understood that in some embodiments channels are not formed or defined in a substrate. For example, as shown in FIG. 15, channel may be defined within a freestanding tube, such as a glass tube.

Microfluidic channels 120 may also perform additional functions, such as facilitating chemical reactions, for example, by the use of catalysts or enzymes immobilized on a wall of the reaction chamber, or introduced through another inlet. The channels may also connect with other components, for example, a detection sensor. Any of the components may be present within system 100 (for example, within a channel) or be fluidly connected to system 100 through one or more outlets. Detection techniques may include any suitable technique for detecting a chemical or biological species. Suitable techniques include measurement of fluorescence (e.g, light, ultraviolet, or infrared), measurement of electrical capacitance, or measurement of other physical properties such as magnetic inductance or radioactivity.

Microfluidic system 100 may be used in any system where manipulation of a species may be desired. Such manipulations may include, but are not limited to, separation, sorting, immobilization, trapping, segregating, filtration, assaying, or magnetic detection.

In one embodiment of the invention, system 100 is used to separate, sort, entrap, or filter different types of particles. In some cases, the particles are separated as a result of their different magnetic susceptibilities. Multiple species, attached to multiple magnetic particles, can be separated or sorted in this fashion. The particle or species may be captured or immobilized by the confined magnetic field. Particles having different magnetic susceptibilities may be attracted by magnetic fields of different strengths. For example, a magnetic field may be strong enough to attract a first particle having a first magnetic susceptibility, yet be unable to attract a second magnetic particle having a second magnetic susceptibility, allowing the first particle to be separated from the second particle. Particles or species immobilized on the magnetic features may also be released and delivered into separate outlet channels, for example, by controlling or deactivating the external magnetic fields.

In certain embodiments of the invention, microfluidic system 100 is used to assay or detect various particles or species. For example, particles having a certain amount of a ferromagnetic substance may be detected or measured by selective application of the magnetic field used to manipulate the particles. Particles immobilized by magnetic features 160 may be detected and quantified. Suitable quantification techniques, include optical detection techniques, or by measurement of the mass, radioactivity, magnetic susceptibility, or other physical properties of the particles. Species 110 or particle 140 may be detected or analyzed based on the strength of the magnetic field used to immobilize the species or particle in the channel, or based on the strength of the magnetic field needed to cause the release of a species or particle immobilized on a magnetic feature.

System 100, including substrate 150 and microfluidic channel 120, may be fabricated by the techniques described above. For example, substrate 150 may be any type of substrate used in integrated circuit applications, such as a microchip. Suitable substrate materials include semiconductor materials (e.g., silicon or GaAs) or polymeric materials, such as polydimethylsiloxane (“PDMS”) or glass. Suitable soft lithography techniques have been described in, for example, Xia, et al., “Soft Lithography [Review],” Angew. Chem. Int. Ed., 37:551-575, 1998. A suitable technique is also described in Example 2.

The function and advantage of these and other embodiments of the present invention will be more fully understood from the example below. The following example is intended to illustrate the benefits of the present invention, but do not exemplify the full scope of the invention.

### EXAMPLE 1

### Micromagnetic System and Method

This example illustrates the ability of a micromagnetic system to manipulate magnetic particles.

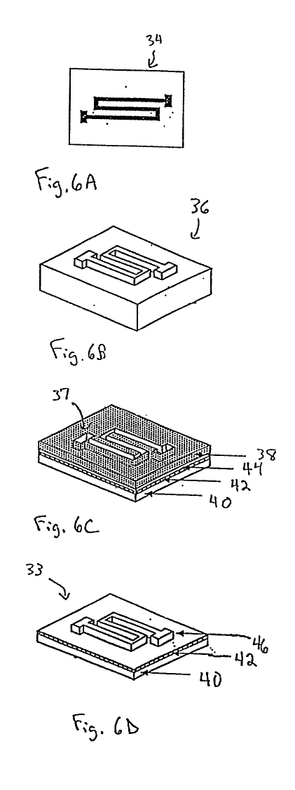

The micromagnetic system included a microchip substrate which was produced using a soft lithography process. FIGS. 6A-6D schematically illustrate the steps of the soft lithography process to form a microchip 33. FIG. 6A shows a CAD design 34 of the microchip. FIG. 6B shows micromolds 36 produced in a rapid prototyping step from CAD design 34. Micromolds 36 were made of polydimethylsiloxane (PDMS). Micromolds 36 were used to form a pattern 37 in a polyurethane layer 38 on a silicon substrate 40 using a microtransfer molding technique (FIG. 6C). Silicon substrate 40 also included a silicon oxide layer 42 and a silver layer 44 formed in succession beneath polyurethane layer 38. Pattern 37 was filled with gold using an electroplating technique. Polyurethane layer 38 was etched using a solution of CH₂Cl₂: CH₃0H: NH₄OH, (100:25:3). Then, a wet chemical etching process was used to remove silver layer 44 using an aqueous solution of 0.1 M Na₂S₂O₃/0.01M K₃ Fe(CN)₆/0.001M K₄Fe(CN)₆. After the etching steps, gold wires 46 were formed on the surface of chip 33. The height of wires 18 was controlled during the electroplating process. Wires 46 had uniform dimensions verified by measurements of a profilometer.

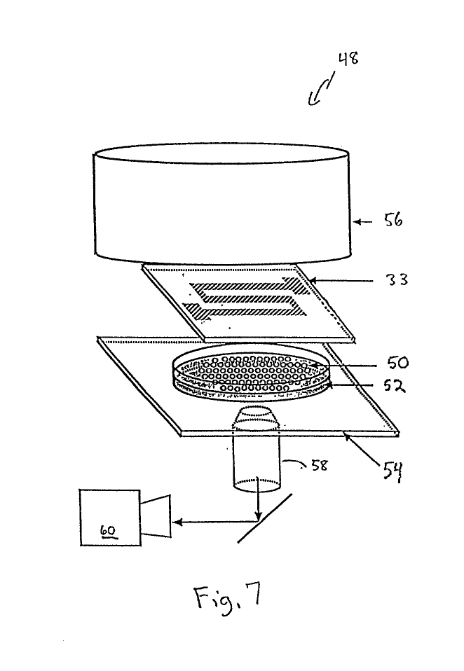

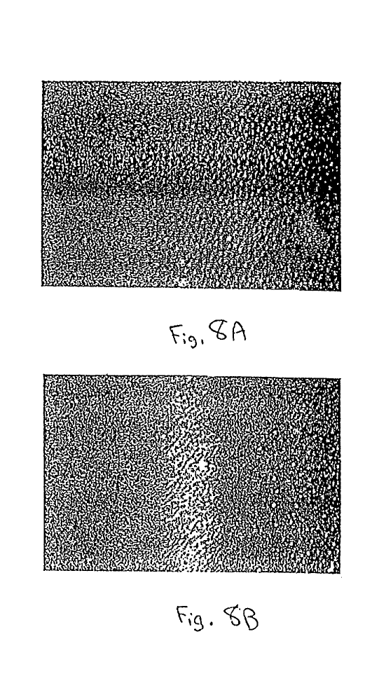

FIG. 7 schematically illustrates a system 48 used to manipulate microbeads 50 using magnetic fields generated by current flowing through wires 46 on chip 33. Microbeads 50 were composed of a magnetite core surrounded by a polystyrene shell (Dynal M-450, manufactured by Dynal, Inc.; Lake Success, N.Y.) and had a diameter of about 4.5 microns. Microbeads 50 were dispersed in a water solution to provide a mixture. The mixture of microbeads 50 and water was confined in a container 52 disposed on a sample holder 54 below chip 33. The distance between the surface of the mixture and chip 33 was between about 100 microns and 500 microns. Though the experiment did not include magnetic beads dispersed on the surface of the integrated chip, it is to be understood that this configuration could also be performed. A permanent magnet 56 was positioned above chip 33 to provide an external magnetic field. System 48 included a lens 58 and CCD camera 60 to record images of microbeads 50.

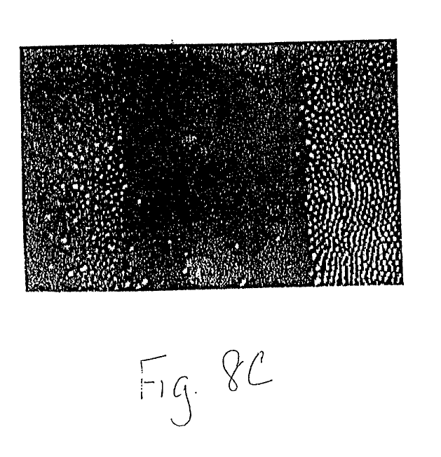

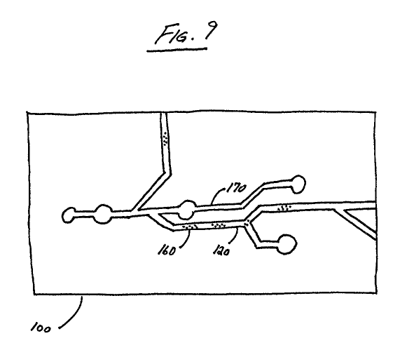

FIG. 8A is a micrograph showing microbeads 50 prior to the generation of a magnetic field. In FIG. 9A, microbeads 50 are dispersed uniformly throughout the mixture.

A current of about 1 A was passed through wires 46 in a first direction to generate a magnetic field. An external magnetic field was superimposed on the field generated by the current carrying wires using permanent magnet 56. FIG. 8B is a micrograph showing the confinement of microbeads 50 in a channel using the magnetic fields.

A current of about 1 A was passed through wires 46 in a second direction opposite to the first direction (as described above in connection with FIG. 8B) to generate a magnetic field. An external magnetic field was superimposed on the field generated by the current carrying wires using permanent magnet 56. The external magnetic field was in the same direction as described above in connection with FIG. 8B. FIG. 8C is a micrograph showing the expulsion of microbeads from a channel using magnetic fields.

The example shows how a micromagnetic system may be used to manipulate magnetic particles. Specifically, the system was used to selectively confine and expel magnetic microbeads within a channel.

### EXAMPLE 2

### Fabrication of Arrays of Nickel Posts using Soft Lithography

This example illustrates one method of producing a microfluidic system having an array of magnetic features. A schematic diagram of this process can be seen in FIG. 12.

Polydimethylsiloxane molds were initially fabricated using a rapid prototyping process (see, for example, Qin, et al., Adv. Mater., 8:917, 1996). The particular molds used in this example were designed to produce a substrate approximately 1.0 mm thick, containing an array of 15 cylindrical posts, with a height of approximately 7 μm and a diameter of approximately 15 μm.

Using microtransfer molding techniques (see, for example, Zhao, et al., Adv. Mater., 8:837, 1996), the polydimethylsiloxane patterns were transferred into features in polyurethane on a silicon wafer coated with layers of titanium and gold. The titanium was about 50 A in thickness and the gold layer was about 500 A in thickness. The features formed in the polyurethane using this molding technique were about 7 μm thick. Next, the polyurethane was cured by exposure the preparation to ultraviolet light from a 450 W medium-pressure Hg vapor lamp for approximately 1 hour. The preparation was placed approximately 1 cm from the lamp.

The electrodeposition of nickel onto the substrate was performed using a nickel sulfamate plating solution. A block of nickel metal was used as the anode. The applied current density was approximately 60 mA/cm² during the electrodeposition process.

After electrodeposition, the polyurethane-resistant layer was removed using a solution of methylene chloride:methanol:ammonium hydroxide having a 100:25:3 ratio by volume. The ammonium hydroxide component was a concentrated solution of 30% ammonia and water by weight.

A polydimethylsiloxane layer was then formed on top of the silicon wafer containing the nickel metal posts. The polydimethylsiloxane layer contained several rectangular microfluidic channels, approximately 50 μm high and approximately 150 μm wide. The polydimethylsiloxane layer and the silicon layer containing the nickel posts were aligned under a microscope using a micromanipulation device.

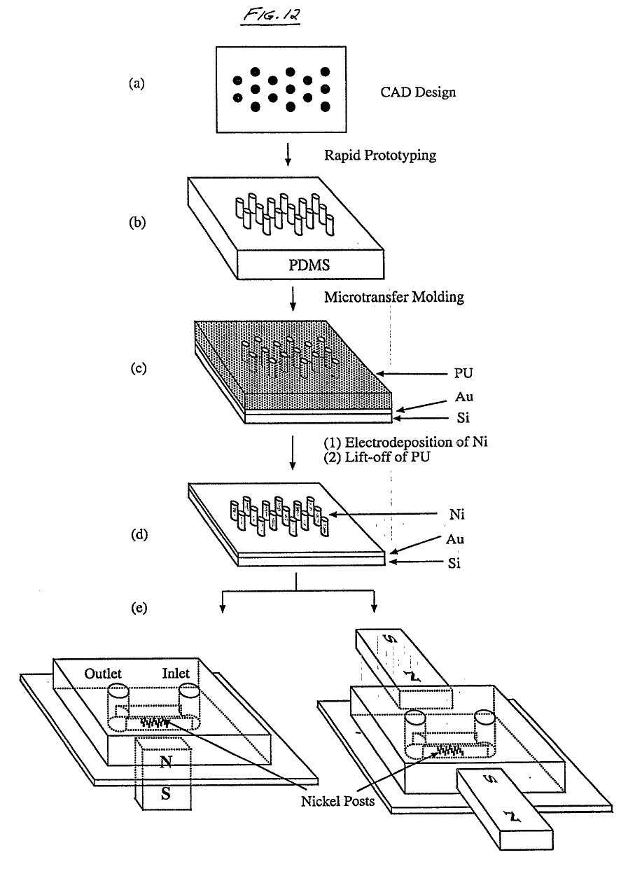

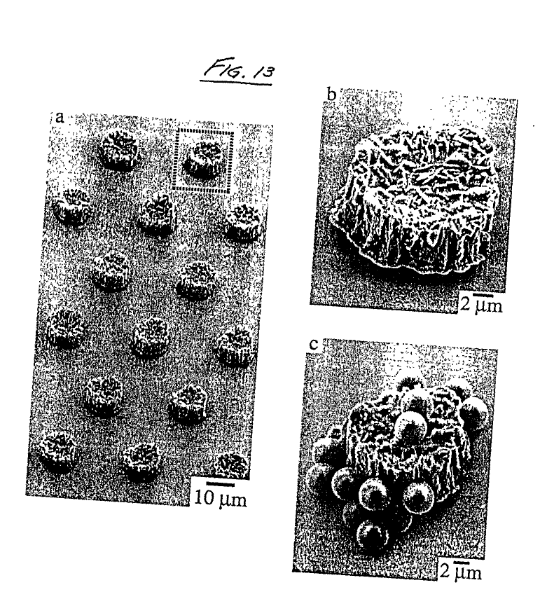

This technique produced nickel posts on a 1.0 mm-thick silicon/ polydimethylsiloxane substrate, within a fluid channel having a height of approximately 50 μm, as can be seen FIGS. 13A and 13B. An array of 15 posts was formed by this technique. Each nickel post was cylindrical, with a height of approximately 7 μm high, and a diameter of approximately 15 μm. The surface roughness of each post was found to be 0.5 to 1.0 μm.

Thus, this example illustrates one technique of producing a polymeric microfluidic system containing a regular array of nickel posts within a channel.

### EXAMPLE 3

### Trapping of Magnetic Beads

In this example, magnetic beads are manipulated in a microfluidic system. This example demonstrates the trapping of magnetic particles by nickel posts using induced magnetic fields.

A suspension of uncoated magnetic beads having a diameter of approximately 4.5 μm was prepared at a concentration of approximately 10⁴ beads/ml. The suspension contained approximately 1% of Triton X-100 by weight. The magnetic bead suspension was passed through an embodiment of the invention, fabricated as previously described under Example 2. The suspension was passed through the microfluidic channel at a flowrate of approximately 2 μl/min.

External magnets were used to induce magnetic fields within the nickel posts. The external magnets used were a pair of neodymium-iron-boron magnets. Sufficient induced magnetic fields could be generated by orienting the magnetic field in one of two directions: axial (the external magnetic field parallel to the axis of the post and perpendicular to the direction of fluid flow) or transverse (the external magnetic field perpendicular to both the axis of the post and the direction of fluid flow). Movement of external magnets around the microfluidic channel were controlled using micromanipulation devices. The highest field strength was observed when the magnets were located approximately 3 mm from the microfluidic channel.

The induced magnetic field created by the nickel posts was found to be strong enough to trap magnetic beads passing by the posts in suspension. Removing the external magnetic field while keeping the flow velocity of the liquid unchanged released the magnetic beads. FIG. 13C shows the magnetic beads trapped by the magnetic posts illustrating one method of immobilizing magnetic beads or particles within a channel using the invention.

### EXAMPLE 4

### Separation of Magnetic Beads from a Solution Containing Both Magnetic and Non-Magnetic Beads

This example illustrates how an embodiment of the present invention can be used to separate magnetic beads from non-magnetic beads.

The uncoated magnetic beads previously described in Example 3 were mixed with non-magnetic dyed beads having a diameter of approximately 6 μm. The aqueous suspension created contained approximately 10⁴ beads/ml of magnetic beads, approximately 10⁴ beads/ml of dyed beads, and approximately 1% by weight of Triton X-100.

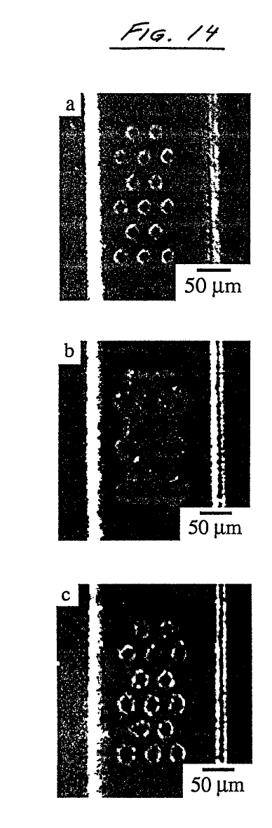

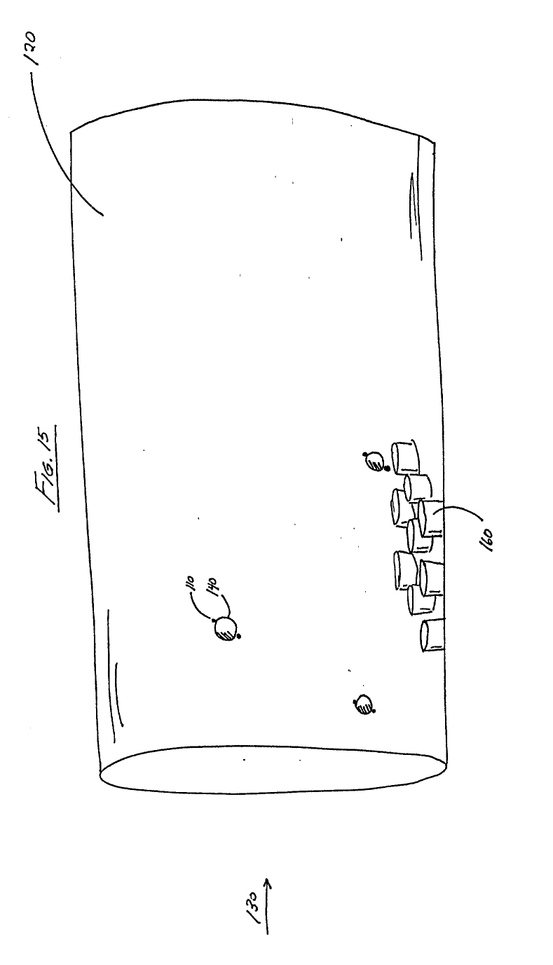

The particle suspension was injected into one embodiment of the invention, fabricated as previously described under Example 2, and shown optically under a microscope in FIG. 14A. After injection of the particle suspension into the microfluidic channel, an external neodymium-iron-boron magnet was positioned near the channel, at a distance of approximately 3 mm from the channel. The particles that are immobilized on the magnetic features are shown in FIG. 14B.

Next, the particle suspension was flushed from the microfluidic channel, using a solution of distilled water containing approximately 1% Triton X-100 by weight. The external magnet was then removed and the suspension, carrying magnetic beads released with the removal of the external magnet, was collected at the outlet. The magnetic features after removal of the particles are shown in FIG. 14C.

Analysis of the collected suspension revealed that more than approximately 95% of the magnetic beads were retained by the invention, thus demonstrating the successful separation of one type of particle from another type of particle.

Those skilled in the art would readily appreciate that all parameters listed herein are meant to be exemplary and that the actual parameters would depend upon the specific application for which the systems and methods of the present invention are used. It is, therefore, to be understood that the foregoing embodiments are presented by way of example only and that, within the scope of the appended claims and equivalence thereto, the invention may be practiced otherwise than as specifically described.

What is claimed:

1. A method of manipulating a biological or a chemical species comprising:
manipulating a biological or a chemical species in a confined space having a maximum dimension of less than 5 cm using a magnetic field.

2. The method of claim 1, wherein the species is attached to a particle comprising a magnetic material.

3. The method of claim 2, wherein the particle includes a coating and a magnetic material core.

4. The method of claim 3, wherein the particle has a size of less than 100 microns.

5. The method of claim 1, wherein the magnetic field is generated by one or more current carrying wires.

6. The method of claim 5, wherein the wires are disposed on a substrate.

7. The method of claim 1, wherein the magnetic field is generated by a magnetizable layer.

8. The method of claim 7, wherein the magnetic field generated by the magnetizable layer is induced in the magnetizable material.

9. The method of claim 1, wherein the confined space is defined on a substrate.

10. The method of claim 9, wherein the confined space comprises a channel defined by local field maxima generated by current flowing through wires formed on the substrate.

11. The method of claim 1, wherein the confined space has a maximum dimension of less than 1 mm.

12. The method of claim 1, wherein the species is suspended in a fluid.

13. The method of claim 1, comprising manipulating the species without fluid flow.

14. The method of claim 1, wherein manipulating the species comprises directing the motion of the species.

15. The method of claim 1, further comprising moving the biological or chemical species by attracting the species with a magnetic field.

16. The method of claim 1, comprising varying the position of the magnetic field by changing the position of local field maxima.

17. The method of claim 1, wherein manipulating the biological or chemical species comprises capturing and confining the biological or chemical species.

18. The method of claim 1, comprising manipulating a first biological or chemical species in a confined space having a maximum dimension of less than 5 cm using a magnetic field to bring the first species in contact with a second biological or chemical species thereby causing a reaction between the first and second species.

19. The method of claim 1, comprising separating a first biological or chemical species from a second biological or chemical species in a confined space having a maximum dimension of less than 5 cm using a magnetic field.

20. A method of manipulating a biological or chemical species comprising:
manipulating a biological or a chemical species on a substrate in the absence of structural boundaries capable of confining the species.

21. The method of claim 20, comprising selectively manipulating the species using a magnetic field.

22. The method of claim 21, wherein the magnetic field is generated by one or more current carrying wire disposed on a substrate.

23. The method of claim 20, wherein the species is attached to a particle comprising a magnetic material.

24. The method of claim 20, wherein the particle has a size of less than 100 microns.

25. The method of claim 21, wherein the magnetic field defines, at least in part, a channel in which the species may move.

26. The method of claim 25, wherein the channel has a maximum dimension of 5 cm.

27. The method of claim 20, wherein the species is suspended in a fluid.

28. The method of claim 20, comprising manipulating the species without fluid flow.

29. The method of claim 20, wherein manipulating the species comprises directing the motion of the species.

30. The method of claim 20, further comprising moving the biological or chemical species.

31. The method of claim 30, comprising moving the biological or chemical species by varying the position of the magnetic field.

32. A method of manipulating a biological or chemical species comprising:
manipulating a biological or a chemical species using a magnetic field generated by one or more current carrying wires.

33. The method of claim 32, wherein the wires are disposed on a substrate.

34. The method of claim 32, wherein the magnetic field defines, at least in part, a channel in which the species may move.

35. The method of claim 34, wherein the channel has a maximum dimension of 5 cm.

36. The method of claim 34, wherein the channel has a maximum dimension of 1 mm.

37. The method of claim 32, wherein the species is attached to a particle comprising a magnetic material.

38. The method of claim 32, wherein manipulating the species comprises directing the motion of the species.

39. The method of claim 32, further comprising moving the biological or chemical species.

40. The method of claim 32, comprising moving the biological or chemical species by varying the position of the magnetic field.

41. A method of manipulating a biological or chemical species comprising:
moving a biological or chemical species in a first direction; and
changing the direction of motion of the biological or chemical species using a magnetic field.

42. The method of claim 41, wherein the magnetic field defines, at least in part, a boundary.

43. The method of claim 41, comprising moving the species in the first direction a distance of less than 5 cm prior to changing the direction of motion.

44. A method of manipulating a biological or chemical species comprising:
manipulating a biological or a chemical species on a substrate in the absence of fluid flow.

45. A microfluidics system comprising:
a substrate including a plurality of wires capable of carrying current to generate magnetic fields that define channels on the substrate; and
a biological or chemical species movable within the channels on the substrate.

46. The microfluidics system of claim 45, wherein the species is attached to a particle comprising magnetic material.

47. The microfluidics system of claim 46, wherein the particle includes a coating and a magnetic material core.

48. The microfluidics system of claim 45, wherein the particle has a size of less than 100 microns.

49. The microfluidics system of claim 45, wherein the substrate has a maximum dimension of less than 5 cm.

50. The microfluidics system of claim 45, wherein the substrate has a maximum dimension of less than 1 mm.

51. The microfluidics system of claim 45, further comprising a fluid disposed on the substrate, the species being flowable through the fluid.

52. The microfluidics system of claim 45, further comprising a voltage source connectable to the plurality of wires.

53. The microfluidics system of claim 45, further comprising an external magnet.

54. The microfluidics system of claim 45, further comprising a magnetizable material layer disposed on the substrate.

55. A microfluidics system comprising:
a channel; and
a feature formed within the channel, the feature capable of generating a magnetic field.

56. The microfluidics system of claim 55, wherein the channel is defined within a substrate.

57. The microfluidics system of claim 56, wherein the substrate comprises a polymer.

58. The microfluidics system of claim 56, wherein the substrate has a largest dimension of less than about 5 centimeters.

59. The microfluidics system of claim 55, wherein the channel is defined within a tube.

60. The microfluidics system of claim 55, wherein the feature comprises a magnetic material.

61. The microfluidics system of claim 60, wherein the magnetic material comprises nickel.

62. The microfluidics system of claim 55, wherein the feature is completely contained within the channel.

63. The microfluidics system of claim 55, wherein the microfluidics system comprises an array of features.

64. The microfluidics system of claim 63, wherein the array has features of at least two different sizes.

65. The microfluidics system of claim 55, wherein the feature comprises a post.

66. The microfluidics system of claim 55, wherein the channel has a width of less than about 1000 micrometers.

67. The microfluidics system of claim 66, wherein the channel has a width of less than about 10 micrometers.

68. The microfluidics system of claim 55, further comprising a fluid within at least a portion of the channel.

69. The microfluidics system of claim 68, further comprising chemical or biological species carried by the fluid.

70. The microfluidics system of claim 69, wherein the magnetic field manipulates the chemical or biological species.

71. The microfluidics system of claim 69, wherein the chemical or biological species are attached to particles.

72. The microfluidics system of claim 71, wherein the magnetic field manipulates the particles.

73. The microfluidics system of claim 69, comprising different types of chemical or biological species.

74. The microfluidics system of claim 73, wherein different types of species are respectively attached to different types of particles.

75. The microfluidics system of claim 73, wherein the system is designed to separate different types of species.

76. A microfluidics system including a channel defined therein, wherein the system is capable of producing a magnetic field confined within the channel.

77. The microfluidics system of claim 76, wherein the magnetic field is an induced magnetic field.

78. The microfluidics system of claim 76, wherein the magnetic field is produced by an electromagnet.

79. A microfluidics system comprising:
a substrate having a channel defined therein; and
a feature formed within the channel, the feature comprising a magnetic material.

80. A method comprising manipulating a chemical or biological species within a channel in a microfluidics system using a magnetic field generated by a feature formed within the channel.

81. The method of claim 80, wherein the chemical or biological species are attached to particles and the particles are manipulated by the magnetic field.

82. The method of claim 81, further comprising manipulating at least two different types of chemical or biological species.

83. The method of claim 82, wherein manipulating the at least two different types of chemical or biological species comprises separating a portion of a first type of chemical or biological species from a portion of a second type of chemical or biological species.

84. The method of claim 80, wherein manipulating the chemical or biological species comprises capturing at least a portion of the chemical or biological species.

85. The method of claim 80, wherein the magnetic field generated by the feature is induced by an external magnet.

86. The method of claim 80, wherein the chemical or biological species are carried within a fluid.

87. The method of claim 80, further comprising manipulating a second species within a second channel in the microfluidics system using a second magnetic field generated by a second feature formed within the second channel.

88. A method comprising generating a magnetic field confined within a microfluidic channel.

89. The method of claim 88, further comprising inducing the magnetic field using a magnetic field external of the microfluidic channel.

90. The method of claim 88, wherein the magnetic field confined within the microfluidic channel is generated without simultaneously generating a second magnetic field confined within a second microfluidic channel.

91. The method of claim 88, wherein the magnetic field is generated by a feature formed within the microfluidic channel.

92. A method comprising manipulating a species in a microfluidics system using an applied magnetic field of less than about 500 gauss within a channel.

93. The method of claim 92, wherein using an applied magnetic field comprises using an applied magnetic field of less than about 100 gauss within the channel.

94. A method of forming a microfluidics system comprising:
forming a channel defined by the microfluidics system; and
forming a feature within the channel, the feature capable of generating a magnetic field.

95. The method of forming a microfluidics system of claim 94, wherein forming a channel comprises forming a channel using soft lithography.

96. A microfluidics system comprising:
a channel;
a feature having a smallest dimension no greater than the smallest dimension of the channel proximate the feature, positioned so as to be capable of generating a magnetic field within the channel.

97. The microfluidics system of claim 96, wherein the channel is defined within a substrate.

98. The microfluidics system of claim 96, wherein the feature comprises a magnetic material.

99. The microfluidics system of claim 96, wherein the microfluidics system comprises an array of features.

100. The microfluidics system of claim 96, wherein the magnetic field manipulates a chemical or biological species.

101. The microfluidics system of claim 100, wherein the chemical or biological species are attached to particles.

102. The microfluidics system of claim 96, wherein the magnetic field is an induced magnetic field.

103. A method comprising:
applying a magnetic field able to manipulate a chemical or biological species within a first channel in a microfluidics system, the magnetic field unable to manipulate a similar species within a second channel in the microfluidics system.

104. The method of claim 103, wherein the second channel is separated from the first channel by no more than 5 centimeters.

105. The method of claim 104, wherein the second channel is separated from the first channel by no more than 1000 micrometers.

106. The method of claim 103, wherein the chemical or biological species are attached to particles and the particles are manipulated by the magnetic field.
